(19) United States
(12) Patent Application Publication
(10) Pub. No.: US 2023/0149440 A1
(43) Pub. Date: May 18, 2023

(54) OPHTHALMIC LIQUID COMPOSITION COMPRISING LOW MOLECULAR WEIGHT LINEAR HYALURONIC ACID

(71) Applicant: WELIFEPHARMA SAGL, Lugano (CH)

(72) Inventors: Pasqua ORESTE, Milano (MI) (IT); Giorgio ZOPPETTI, Milano (MI) (IT)

(21) Appl. No.: 17/918,652

(22) PCT Filed: Apr. 16, 2021
(86) PCT No.: PCT/IB2021/053170

§ 371 Date: Oct. 13, 2022

(30) Foreign Application Priority Data
Apr. 17, 2020 (IT) 102020000008299

(51) Int. Cl. A61K 31/728; A61P 27/04; A61K 47/02; A61K 9/00

(52) U.S. Cl. CPC A61K 31/728; A61K 9/0048; A61K 47/02; A61P 27/04

(57) ABSTRACT

The present invention is related to an ophthalmic liquid composition, useful for example as moisturizer, comprising as the active principle, linear low molecular weight hyaluronic acid (LMWHA).

### BACKGROUND OF THE INVENTION

Hyaluronic acid (HA) is a natural linear polysaccharide belonging to the family of glycosaminoglycans, constituted by a disaccharide repetition formed by glucuronic acid β 1-4 linked to N-acetylglucosamine. It is ubiquitously present in the living organisms and in the last years its application in the pharmaceutical and cosmetic fields as component in several formulations has gained increased attention.

Today hyaluronic acid is industrially produced by fermentation and, depending on the polymeric chain length, it can be distinguished in high molecular weight HA (HMWHA) and low molecular weight HA (LMWHA), having a molecular weight from 3,000 to 250 kDa and below 250 kDa respectively. These two categories have different characteristics and functionalities and are used to reach different targets.

As demonstrated by S.Misra et al., Frontiers of Immunology, 2015, 6, Article 201, the chains of LMWHA with molecular weight from 3 to 6.5 kDa induce angiogenesis in the chick corneal assay, and those included from 6 to 20 kDa induces inflammation on dendritic cells.

Many commercial ophthalmic formulations as eye drops containing HA, exploit the wound healing and moisturising properties of HA, and use high or medium-high molecular weight HA. Unfortunately, the high or medium-high molecular weight of HA limits the possibility to obtain suitable concentrated solutions, the concentration being usually about 0.1% of HA and only rarely reaches 0.3%, due to the solubility limit of said polymers and to the excessive viscosity of the formulations containing them.

Applicant believes that the use of LMWHA, if available in a restricted range of molecular weight which guarantees consistent performances, would allow the preparation of eye drops with higher concentrations of active ingredient without influencing the viscosity of the final solution nor causing problems due to its solubility limits.

Therefore, the availability of a novel liquid ophthalmic compositions comprising LMWHA with a specific mean molecular weight within a controlled, specific range of molecular weight and without degradation by-products is still needed.

### OBJECTS OF THE INVENTION

An object of the present invention is to provide an ophthalmic liquid formulation comprising a low molecular weight hyaluronic acid (LMWHA).

Further object of the invention is the use of said liquid ophthalmic composition in eye drop diseases and/or as an eye drop composition to be used, for instance, in the treatment of dry eye symptoms such as pain, hitching, ocular burning or foreign body in the eye sensation. Such symptoms could be of any origin for instance they may be caused by external factors, such as air conditioning, pollution, air travels, video terminal working, refractive surgery, contact lens use, or caused by pathologies, like the Meibomio gland disfunction, and the like. Thanks to its properties, LMWHA moisturise, lubricate and protect the ocular surface.

### BRIEF DESCRIPTION OF THE FIGURES

FIG. 1 shows the graph of the viscosity correlated to the molecular weight during the depolymerisation reaction in 0.1 N HCl at 60° C. of Example 1.

FIG. 2 shows an example of standard curve of the HPLC analysis using standards with absolute molecular weights.

FIG. 3 shows the comparison between the HPLC retention time of the product of Example 1 and of the standard having 100 kDa molecular weight.

### DESCRIPTION OF THE INVENTION

The subject-matter of the present invention is a liquid ophthalmic composition comprising a linear, low molecular weight hyaluronic acid (LMWHA) and at least one ophthalmic acceptable carrier.

According to a preferred embodiment, the low molecular weight hyaluronic acid (LMWHA) is the only active ingredient present in the composition of the present invention.

According to the present invention, unless otherwise indicated, molecular weight (MW) means weight average molecular weight (MW_{w}).

Even if not indicated, the LMWHA according to the invention is linear, where “linear” means that the LMWHA according to the invention is not “cross-linked”. “Cross-linked” is referred to a HA in which the polysaccharide chains are linked together through covalent bonds by a cross-linking agent. The cross-linked hyaluronic acid, defined as the pool of covalent bond linked chains obtained by a cross-linking agent, forms a highly viscous water-based gel (>50,000 mPa•s), insoluble in water which does not allow the use in ophthalmic devices, in particular, but not limited to, multidose devices.

“Ophthalmic acceptable” here means that such a composition is suitable to the administration to eye and does not induce damages or disorders.

Such liquid ophthalmic composition preferably comprises an amount of the described above LMWHA from 0.5% to 2% weight/volume, more preferably from 1 to 2%, preferably from 1.1 to 2%, more preferably from 1.2 to 2%, advantageously from 1.5% to 2% weight/volume.

The compositions of the invention containing said concentrations of LMWHA are preferably characterised with a viscosity range from 1.5 mPa•s to 30 mPa•s, more preferably from 5 mPa•s to 15 mPa•s.

“Low molecular weight hyaluronic acid” herein indicates the polymer, preferably in a salt form, more preferably as its sodium salt, having a mean chain length such as the mean molecular weight ranges from 90 kDa to 120 kDa, preferably from 95 kDa to 110 kDa, more preferably of around 100 kDa, with a polydispersion <4 preferably 2.5. According to a preferred embodiment, the invention relates to a liquid ophthalmic composition comprising linear, low molecular weight hyaluronic acid having a mean molecular weight ranges from 90 kDa to 120 kDa, said hyaluronic acid being present in an amount from 0.5% to 2% weight/volume, more preferably from 1 to 2%, preferably from 1.1 to 2%, more preferably from 1.2 to 2%, advantageously from 1.5% to 2% weight/volume, and at least one ophthalmic acceptable carrier, for its use in the treatment of eye diseases and/or as eye drops and/or as artificial tears. In all the preferred embodiments of the invention, the LMWHA is sodium hyaluronate.

The composition of the invention also comprises a liquid carrier at pH 7.5 constituted by a buffer solution as the ones conventionally used in the ophthalmic field, preferably borate or phosphate buffers, boric acid or the combination of sodium borate and boric acid.

The composition of the present invention can be used as artificial tear to treat the dry eye syndrome, as moisturiser and/or to relief the symptoms derived from the poor fluid present in the conjunctiva, in humans or animals. The composition of the invention is also useful to contact lens wearing subjects.

It is herein also disclosed a process for the preparation of linear LMWHA, comprising the depolymerisation of linear HMWHA, in particular a controlled depolymerisation of HMWHA in acidic conditions, preferably at pH below 2, advantageously at about pH 1-1.5, more preferably at pH 1.

The depolymerisation is performed starting from a linear HMWHA, preferably with a molecular weight comprising from 1 and 2 MDa.

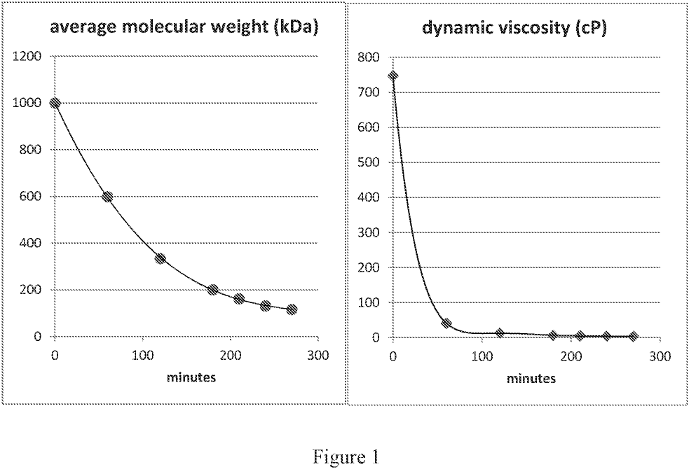

During the depolymerisation reaction of HMWHA, together with the decrease of the molecular weight of HA a decrease of viscosity is observed according to a non-linear kinetic. The viscosity data, actually, after a rapid decrease at the initial stage of the depolymerisation reaction, reach a “steady state”, in which both the molecular weight and the viscosity proceed to decrease but with a much lower speed (see FIG. 1 referring to Example 1).

It was also observed that the possibility to reach the condition of “steady state” in the depolymerisation kinetic depends on the HA initial concentration and on the hydrochloric acid concentration used in the reaction. Surprisingly and in contrast to the literature data, the reaction proceeds efficiently also at pH below 2, and preferably around pH 1.

Said decrease in the kinetic rate is herein specifically pursued and it is very useful because, taking into account the direct relationship between the viscosity of the solution and the chains molecular weight of HA, it permits to better control the final molecular weight and a consequent better reproducibility of the process.

In order to obtain a product with a specific mean molecular weight within a given, restricted range, the viscosity of the reaction can be controlled stepwise and the reaction can be stopped when the viscosity of the sample reaches values comprised in a specific viscosity range, from about 4 to about 5 mPa•s, values that are directly correlated to the values of the polymeric chain length of the desired molecular weight. Therefore, the reaction can be stopped by neutralisation, for example adding a solution of a base, like NaOH until pH 7, and the product may be isolated according to the methods known to the skilled in the art.

The product can be isolated from the solvent by filtration, performed, for example, but without limitations, using an filtration membrane, for example with a 10 kDa cut-off, then making the polymer prepcipitating with an appropriate solvent, preferably ethanol, acetone and/or isopropanol. The final product can be obtained by drying, for example keeping the product in an oven, for example at 40° C. for the needed time, for example for a few hours, such as about 4 hours.

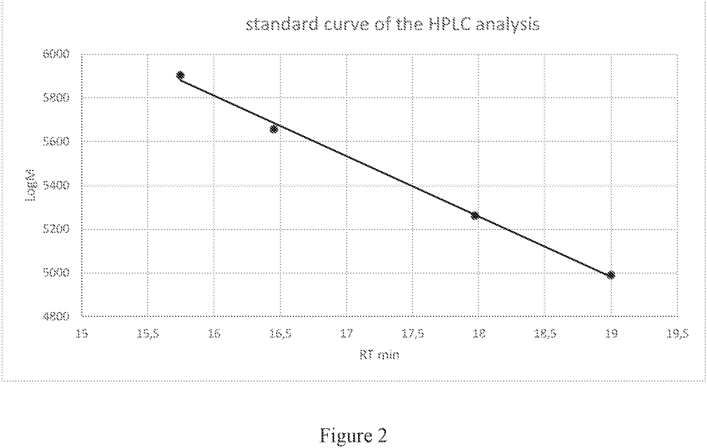

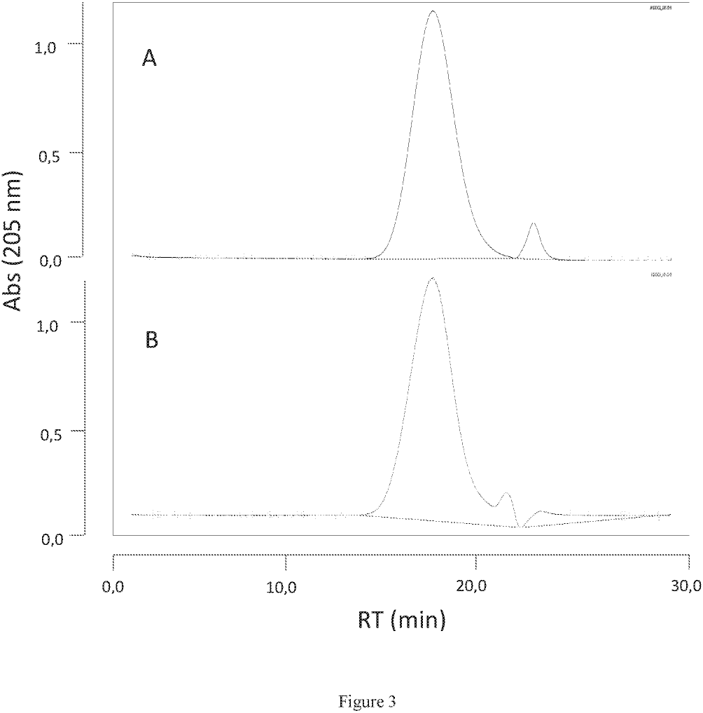

The molecular weight of the HA obtained according to the reaction herein disclosed, can be measured according to known methods for example by a HPLC method using HA molecular weight standards with different chain lengths, characterised by an absolute specific mean molecular weight. Such HPLC method can be preferably linked to different detectors, in particular to measure the viscosity, IR and/or UV. The peak of the sample under examination is compared with the calibration curve obtained with the HA standards characterised by an absolute specific molecular weight to evaluate the sample molecular weight (see FIGS. 2 and 3 with reference to Example 1).

It is also disclosed a process for the preparation of linear LMWHA having a molecular weight ranging from 90 kDa to 120 kDa, preferably from 95 kDa to 110 kDa, more preferably around 100 kDa, by a depolymerisation comprising:
a) dissolving linear HMWHA in water, at a concentration of about 2% (p/v):
b) heating the solution and add a strong acid to obtain pH 1-1.5;
c) measuring the viscosity of the solution at different times;
d) stopping the depolymerisation by adding a strong base when the viscosity reaches a value of 4-5 mPa•s;
e) isolating the LMWHA thus obtained.

Step (a) is preferably performed in water, at room temperature, where “room temperature” means a temperature of 20-25° C., under stirring.

In step (b) the solution is heated at temperature of 40-80° C., preferably about 60° C. The strong acid is preferably hydrochloric acid. According to a preferred embodiment, the acid is 0.1-0.3 N hydrochloric acid, advantageously 0.2 N. The pH of the solution preferably is about 1.

In step (c) the viscosity is controlled by known methods. Some examples are reported in the experimental section below. The strong base preferably is an alkaline-metal hydroxide, advantageously sodium hydroxide. The base is added until the solution is neutral, around pH 7.

In step (d) the LMWHA is isolated according to conventional techniques. Some examples are reported in the experimental section below.

The LMWHA obtained according to the process described above, has a reduced range of the mean chain length, so that the mean molecular weight is from 90 kDa to 120 kDa, preferably from 95 kDa to 110 kDa, more preferably around 100 kDa.

The molecular weight distribution index of LMWHA obtained by the process herein disclosed, calculated as weight average molar mass (Mw) divided by the number average molar mass (Mn), is less than 4, preferably less than 3, more preferably less or about 2.5.

The process for the preparation of LMWHA is characterised by the fact that it minimises the presence of very low molecular weight chains below 20 kDa in the final product. Such very low molecular weight chains actually are known in the literature as having pro-inflammatory properties.

The LMWHA having a specific mean molecular weight with batch-to-batch consistency.

The LMWHA which is used in the compositions of the invention, shows the following features:
weight average molecular weight (Mw) from 90 kDa to 120 kDa, preferably from 95 kDa to 100 kDa, more preferably about 100 kDa;.
linear chains (not cross-linked);
chains with a molecular weight below 20 kD less than 5% (p/p) of the total chains, measured by HPLC in the conditions described in the experimental section below;
distribution index below 4, preferably below 2.5.

Moreover, a subject-matter of the present invention is an ophthalmic composition obtained by dissolving the LMWHA herein disclosed in a suitable solvent, preferably selected among the ophthalmic acceptable buffers, preferably phosphate buffer, borate buffer and boric acid, in a concentration range from 0.5 to 2% weight/volume, preferably from 1.1 to 2%, more preferably from 1.2 to 2%,preferably from 1.5 to 2%. Such high concentrations of hyaluronic acid in a liquid composition for ophthalmic use can be obtained only for the molecular weight characteristics of the LMWHA herein disclosed which, as described, has a low molecular weights and a restricted molecular weight range. The presence of chains of higher molecular weight, effectively would increase excessively the viscosity, making such compositions impossible to use in the ophthalmic liquid applications. On the other hand, the presence of high percentages of very low molecular weight chains could induce toxicity issues due to their pro-inflammatory action.

Preferably, the LMWHA herein disclosed is the sole active principle of the composition of the invention but, if desired or needed, other actives and/or conventional excipients can be added to such composition.

The use of the LMWHA herein disclosed as moisturiser agent and/or as artificial tears and/or for treating dry eyes constitutes a further subject-matter of the invention.

The use of the LMWHA herein disclosed for the preparation of a liquid ophthalmic composition, for example useful as moisturiser agent and/or as artificial tears and or for treating dry eyes constitutes a further subject-matter of the invention.

The liquid ophthalmic composition of the invention for its use in eye diseases and/or as eye drops, and/or as artificial tears to be used for example in the treatment of the dry eye symptoms such as pain, itch, ocular burning or foreign body sensation, represents another subject-matter of the invention.

According to another aspect, a further subject-matter of the invention is a method for the treatment of eye diseases, comprising the dry eye and/or as eye drops and/or as artificial tears to be used for example in the treatment of the dry eye symptoms such as pain, itch, ocular burning and foreign body sensation, which comprises administering an effective amount of LMWHA herein disclosed or of the liquid ophthalmic composition of the invention to a subject, preferably to mammals, preferably to human being.

The LMWHA per se and the process for its preparation as herein disclosed are not subject-matter of the invention. The invention will be described in the experimental section below, as illustrative and non-limiting examples.

### Experimental Section

Even if not indicated, viscosity was measured by a rotational viscometer (Brookfield DVII-Pro) equipped with a small sample adapter with the spindle SC4-18 at 20° C. at 0.1 rpm.

Even if not indicated, HPLC was performed in the following conditions: isocratic system with 0.15 M NaCl buffer pH 7, TSK 6000 column with guard column, run time of 30 minutes and flux of 0.5 ml/min and UV detector at 205 nm.

### Example 1 Preparation of LMWHA for Use According to the Invention

5 g of HMWHA, mean molecular weight 1.0 MDa, were dissolved in 250 ml of water at room temperature under stirring. The solution was brought to 60° C., then 250 ml of 0.2 N HCl (pH 1) were added and the solution was kept under stirring. When the solution reached 60° C. the first sample was collected as time zero sample (t₀). Other samples were collected at given time intervals, as described in Table 1, and the measure of the viscosity of the solution and mean molecular weight of the hyaluronic acid were performed. The measure of the viscosities was performed by a rotational viscometer (Brookfield DVII-Pro) equipped with a small sample adapter with the spindle SC4-18 at 20° C. at 0.1 rpm, taking 16.5 g of the solution and bringing it to 25 ml of 0.25 M phosphate buffer (pH 8). The molecular weight of the polymer in solution was measured by HPLC in isocratic system with 0.15 M NaCl buffer pH 7, TSK 6000 column with guard column, run time of 30 minutes and flux of 0.5 ml/min and UV detector at 205 nm.

The reaction was stopped after 270 min (t₂₇₀); the viscosity of the last sample collected was 4 mPa•s. The sample t₂₇₀ was filtered by a 10,000 Da membrane (Millipore Prepscale) bringing the solution 10 times more concentrated and making a precipitation of the product with 3 volumes of ethanol. The precipitate was kept at 40° C. for 4 hours. The polymer thus isolated showed a mean molecular weight measured by HPLC of 116 kDa, and a distribution index of 1.63, calculated as weight average molecular weight (M_{w}) divided by the average number molecular weight (Mₙ).

TABLE 1

viscosity data and mean molecular weight of the samples collected at different times during the depolymerization reaction (FIGS. 1-3 ).

| Sample | Time (minutes) | Viscosity (mPa•s) | Mean molecular weight Mw (kDa) |
|---|---|---|---|
| t₀ | 0 | 746.8 | 1,000 |
| t₆₀ | 60 | 41.27 | 598.5 |
| t₁₂₀ | 120 | 12.87 | 334.4 |
| t₁₈₀ | 180 | 6.63 | 200.6 |
| t₂₁₀ | 210 | 5.22 | 162.0 |
| t₂₄₀ | 240 | 4.46 | 131.6 |
| t₂₇₀ | 270 | 4.00 | 116.3 |

### Example 2 Preparation of LMWHA for Use According to the Invention

39.7 g of HMWHA, mean molecular weight 1.4 MDa, were solubilised in 1,998 litres of water. The starting sample of HA in powder was added stepwise under stirring and the full dissolution was obtained in 6 hours. The solution was kept overnight at room temperature, then the temperature was brought to 60° C. under stirring. When the temperature of 60° C. was reached, 1,998 litres of 0.2 N HCl (pH 1) were added to the solution keeping the temperature at 60° C. and stirring. Samples of 16.5 g of solution were collected at different times and diluted to 25 ml with 0.25 M phosphate buffer (pH 8). The viscosity was measured by a rotational viscometer (Brookfield DVII-Pro) equipped with a small sample adapter with the spindle SC4-18 at 20° C. and 0.1-50 rpm. After 180 minutes of reaction, the sample collected had a viscosity of 4.6 mPa•s. The reaction was stopped by neutralisation with 1 N NaOH (pH 7). The product was filtered by a 10,000 Da membrane (Millipore Prepscale) to concentrate the solution 10 times and then precipitated with 3 volumes of ethanol. The precipitate was dried at 40° C. for 4 hours. The polymer isolated showed a mean molecular weight measured by HPLC of 95 kDa, and a distribution index of 2.55, calculated as weight average molecular weight (M_{w}) divided by the average number molecular weight (Mₙ).

### Example 3 Preparation of LMWHA for Use According to the Invention

60 g of HMWHA, mean molecular weight 1.4 MDa, were solubilised in 3 litres of water. The starting sample of HA in powder was added stepwise at 40° C. under stirring and the full dissolution was obtained in 6 hours. The solution was kept overnight at room temperature, then the temperature was brought to 60° C. under stirring. When the temperature of 60° C. was reached, 3 litres of 0.2 N HCl (pH 1) were added to the solution keeping the temperature at 60° C. under stirring.

Samples of 16.5 g of solution were collected at different times and diluted to 25 ml with 0.25 M phosphate buffer (pH 8). The viscosity was measured by a rotational viscometer (Brookfield DVII-Pro) equipped with a small sample adapter with the spindle SC4-18 at 20° C. and 0.1 rpm.

After 140 minutes of reaction, the sample collected had a viscosity of 4.32 mPa•s. The reaction was stopped by neutralisation with 1 N NaOH (pH 7). The product was filtered by a 10,000 Da membrane (Millipore Prepscale) to concentrate the solution 10 times and precipitating the product with 3 volumes of acetone and washed with isopropanol. The precipitate was dried at 40° C. for 4 hours. The polymer isolated showed a mean molecular weight measured by HPLC of 120 kDa, and a distribution index of 2.45, calculated as weight average molecular weight (M_{w}) divided by the average number molecular weight (Mₙ).

### Example 4 - Measure of the Viscosity of LMWHA (for Use According to the Invention) Containing Solutions

The product obtained according to the depolymerisation reaction described in Example 1, after drying, is dissolved in water at different concentrations.

The viscosity of the solutions is measured by a rotational viscometer Brookfield (DVII Pro) equipped with a small sample adapter and a spindle SC4-18 at 20° C. and 0.1 rpm. The results obtained are reported in Table 2.

Table 2: viscosity of the aqueous solutions at increasing concentration of LMWHA with chains mean molecular weight of 100 kDa.

| concentration (% weight/volume) | viscosity (mPa•s) |
|---|---|
| 4 | 240 |
| 6 | 750 |
| 8 | 2,700 |
| 10 | 6,700 |

### Example 5 - Preparation of an Ophthalmic Composition With 2% (w/v) LMWHA

1.5 g of LMWHA, mean molecular weight (mw) 100 kDa, prepared as described in example 1, were dissolved in 75 ml of 0.08 m phosphate buffer (pH 7.5) under stirring, obtaining a 2% solution of the polymer. The viscosity was measured by a rotational viscometer Brookfield (DVII Pro) equipped with a small sample adapter and a spindle SC4-18 at 20° C. and 0.1 rpm. The dynamic viscosity of the 2% solution of LMWHA with mean molecular weight of 100 kDa was 27.59 mPa.s.

### Example 6 - Preparation of an Ophthalmic Composition With 1.5% (w/v) LMWHA

25 ml of the solution obtained in Example 5 were diluted with 8.3 ml of 0.08 M borate buffer (pH 7.5) under stirring, obtaining a 1.5% solution of the polymer. The viscosity was measured by a rotational viscometer Brookfield (DVII Pro) equipped with small sample adapter and a spindle SC4-18 at 20° C. and 0.1 rpm. The dynamic viscosity of the 1.5% solution of LMWHA with mean molecular weight of 100 kDa was16.80 mPa.s. Said solution was filtered with a 0.22 µm filter and filled in a multidose device suitable for ophthalmic administrations.

### Example 7 - Preparation of an Ophthalmic Composition With 1% (w/v) LMWHA

25 ml of the solution obtained in Example 5 were diluted with 25 ml of 0.08 M borate buffer (pH 7.5) under stirring, obtaining a 1% solution of the polymer. The viscosity was measured by a rotational viscometer Brookfield (DVII Pro) equipped with small sample adapter and a spindle SC4-18 at 20° C. and 0.1 rpm. The dynamic viscosity of the 1% solution of LMWHA with mean molecular weight of 100 kDa was 7.80 mPa.s. This solution was filtered by a 0.22 µm filter and filled in a multidose device suitable for ophthalmic administrations.

1. A method for treating eye diseases, said method comprising:
administering a liquid ophthalmic composition comprising a linear, low molecular weight hyaluronic acid having a mean molecular weight ranges from 90 kDa to 120 kDa, said hyaluronic acid being present in an amount from 0.5% to 2% weight/volume of the composition, and at least one ophthalmic acceptable carrier.

2. The method of claim 1, wherein said hyaluronic acid is present in an amount from 1.1% to 2% weight/volume of the composition.

3. The method of claim 2, wherein said hyaluronic acid is present in an amount from 1.5% to 2% weight/volume of the composition.

4. The method of claim 1, wherein said hyaluronic acid has a mean molecular weight from 95 kDa to 110 kDa.

5. The method of claim 4, wherein said hyaluronic acid has a mean molecular weight of about 100 kDa.

6. The method of claim 1, wherein said carrier is selected from ophthalmic acceptable buffers.

7. The method of claim 1, wherein said hyaluronic acid is the sole active ingredient contained in said composition.

8. A liquid ophthalmic composition comprising
linear, low molecular weight hyaluronic acid having a mean molecular weight ranges from 90 kDa to 120 kDa, said hyaluronic acid being present in an amount from 1.1% to 2% weight/volume of the composition, and
at least one ophthalmic acceptable carrier.

9. The composition of claim 8, wherein said hyaluronic acid is present in an amount from 1.5% to 2% weight/volume of the composition.

10. The composition of claim 8, wherein said hyaluronic acid has a mean molecular weight from 95 kDa to 110 kDa.

11. The composition of claim 10, wherein said hyaluronic acid has a mean molecular weight of about 100 kDa.

12. The composition of claim 8, wherein Composition said carrier is selected from ophthalmic acceptable buffers.

13. The composition of claim 8, wherein said hyaluronic acid is the sole active ingredient contained in said composition.

14. The composition of claim 8, having a viscosity ranging from 1.5 mPa-s to 30 mPa-s at 20° C.

15. The composition of claim 10, having Composition a viscosity ranging from 5 mPa-s to 15 mPa-s, at 20° C.

16. The method of claim 6, wherein said ophthalmic acceptable buffers-are selected from phosphate buffer, borate buffer and boric acid.

17. The composition of claim 12, wherein said ophthalmic acceptable buffers are selected from phosphate buffer, borate buffer and boric acid.

18. The composition of claim 8 in form of eye drops or artificial tears.
